(19) United States
(12) Patent Application Publication
(10) Pub. No.: US 2003/0167008 A1
(43) Pub. Date: Sep. 4, 2003

(54) Malignancy detection apparatus

(76) Inventors: Gary Rogers, Fleggburgh (GB); Mark Hugh Shopland, Norwich (GB)

Correspondence Address: Alexis Barron, Synnestvest & Lechner, 2600 Market Tower, 1101 Market Street, Philadelphia, PA, 19107-2950, US

(21) Appl. No.: 10/258,627

(22) PCT Filed: Apr. 20, 2001
(86) PCT No.: PCT/GB01/01776

(30) Foreign Application Priority Data
Apr. 25, 2000 (GB) 0009863.2

(51) Int. Cl. A61B 5/05

(52) U.S. Cl. 600/474

(57) ABSTRACT

Melanoma is currently a major concern to the general public, and referrals of patients to hospitals by doctors in general practice have multiplied in number. It would be advantageous if a system were available for use by such doctors that was capable of reliably testing skin lesions to determie whether or not a particular lesion was malignant. The present invention proposes such a system using an infra-red sensor in a housing (10) that is scanned, in a measurable, position-aware manner, over the skin to sense the skin's temperature in the area of and around the suspect lesion. The sensor's output being recorded and then displayed to provide a profile of the temperature along the scanned track, from which profile can be deduced something about the likely nature of the lesion.

This invention is concerned with malignancy detection apparatus, and relates in particular to a system for detecting skin and sub-skin malignancies, especially but not exclusively melanoma.

The expression “sub-skin” malignancies used herein means malignancies which lie immediately beneath the skin, such as sub-skin growths of mole-like form. The invention is not intended to include the detection of deep seated malignancies, in respect of which the signal-processing techniques hereinafter described would in general be inapplicable.

Melanoma is a highly malignant form of skin cancer which manifests itself as a mole-like lesion on the skin. However, at present a cancerous lesion is not readily distinguishable from a benign mole without clinical examination at a hospital, and after histological examination. Somewhat analogously, some forms of sub-skin breast cancer are not readily distinguishable from sub-surface cysts, and require removal and histological examination for proper identification.

Melanoma, particularly, is currently a major concern to the general public, and referrals of patients to hospitals by doctors in general practice have multiplied in number to such an extent that in many hospitals services are overburdened with clinical examinations for melanoma and analogous cancers to the detriment of other services which have to be carried out by the same personnel. It would therefore be highly advantageous if a system were available, for use by doctors in general practice, that was capable of reliably testing skin lesions to determine, at least in some cases, whether or not a particular lesion was malignant, thereby reducing the number of hospital referrals.

The present invention proposes such a system, which is based upon the somewhat surprising fact that skin and sub-surface malignancies of the relevant sort tend to be considerably hotter than the surrounding tissue, and so ought to be detectable simply by determining the relative temperatures of the skin in and around the suspect area. More specifically, the invention suggests apparatus for carrying out this temperature determination, the apparatus using an infra-red sensor that is scanned, in a measurable, position-aware manner, over the skin to sense the skin's temperature in the area of and around the suspect lesion, the sensor's output being recorded and then displayed to provide a profile of the temperature along the scanned track, from which profile can be deduced something about the likely nature of the lesion.

In one aspect, therefore, the invention provides a system for detecting skin and sub-surface malignancies, the system comprising:

an infra-red sensor positionable adjacent the skin, at a given distance above the skin, to detect infra-red radiation emanating from the skin;

means for traversing the sensor in a measurable manner along a line across the skin;

means for sequentially recording the output of the sensor, and thus infra-red radiation incident on the sensor, at a series of points along the line; and

means for displaying a profile of the temperature along the sensed line over the lesion, from which profile there can be drawn a conclusion about the presence or absence of a malignancy.

The invention uses an infra-red sensor to detect infra-red radiation emanating from the skin. The sensor can be of any suitable type, and can measure the infra-red (IR) radiation in any convenient way. It can be a thermopile, but it can also be a focal plane detector, a microbolometer or a radiometer.

If desired, an IR-transparent optical means, such as a germanium lens, can be associated with the sensor to focus the infra-red radiation on to it.

The invention's sensor is positionable adjacent the skin, at a given distance above the skin. A knowledge of the sensor height above the skin is important in order to permit there being made an accurate assessment of the temperature—and specifically of the relative temperature—of the area over which the sensor is travelling compared with that of the adjacent areas. Conveniently the sensor is held by a housing itself positioned a small fixed (and known) but adjustable distance above the skin surface, there being means for guiding the sensor/housing to maintain the required distance above the skin as it traverses the lesion (the area where there has been observed the suspect melanoma). This guide means is advantageously a skin touch rod mounted relative to which the sensor is adjustably mounted—most preferably the sensor is mounted to the traversing means with some appropriate degree of freedom of movement normal to the traversing direction, whereby its height can be altered as thought best.

The sensor height adjustment arrangement may need to take into account lesions—protruding moles, for instance—that are significantly higher than the level of the skin, and therefore the device should have an adjustment possible on the sensor housing arrangement to cope with varying heights of lesion, possibly on a continuous, on-going and automatic basis as the lesion traverse takes place.

The height adjustment arrangement can take various forms. In one embodiment the sensor housing is adjustable prior to use, and its height is set for the duration of scanning. The User judges the height above normal skin of the lesion under study, and sets the sensor height accordingly and appropriately (with a knowledge of the type of sensor being employed in the housing). Different types of sensors—even different samples of the same type of sensor—vary in their tolerances and ability to accurately measure temperature at different focal lengths. In another embodiment the sensor housing automatically adjusts itself so that the distance between sensor and skin surface remains constant, utilising an automatic focus system to ensure that the sensor is optimally focused on the skin surface. The focusing system can conveniently be of the type employed in cameras and video recorders, based on either modulated infra-red or ultrasound emissions to measure distance from sensor to skin.

The system of the invention includes means for traversing the sensor in a measured manner along a line across the skin. The traversing may be effected using a hand-driven system or a powered, self-propelled one, but the important point is that it be a measured one—that is, the position along the line is known, so that the system output can accurately provide temperature as a measure of distance travelled (and thus position). It will be appreciated, therefore, that the means for traversing the sensor along the line across the skin must be such as to effect that traverse in a manner that is practical, purposeful, automated and repeatable, and as indifferent as possible to to operator error—and thus that the means is one where the sensor position, both in height above the skin and in distance travelled, and the distance between sensor positions, is known at all times.

Though other mechanisms are possible—such as lead screws, or skids, or rack-and-pinion, or toothed wheel and ratchet—the traversing means is most conveniently simply one or more wheel to which the sensor and its associated equipment is suitably mounted (and as discussed further hereinafter this wheel can advantageously be involved in the measurement of the distance the sensor travels). The sensor can be housed either in line with the traversing means or parallel thereto; in the former case the sensor is looking at the skin traversed by the means, while in the latter it looks at the skin to one side of the means (so that in use the means will traverse skin adjacent to the suspect skin lesion whilst the sensor moves over the skin lesion itself).

The line which the sensor traverses may, obviously, be any length of and shape/direction of line, but as might be expected the traversed line is most conveniently a straight line extending from a point just at one side of the lesion to a point just at the other side. However, from the point of view of correctly assessing the results it is important that the start and finish positions be know. And in order to know how far from the lesion the scan starts, the device needs to be able to measure a fixed distance from the lesion. To achieve this the device preferably includes measuring means that gauges the distance from where the scan starts to the lesion. This measuring means can be of any form, and though a simple ruler, provided as an integral part of the system apparatus, is quite satisfactory, there are other possibilities. For instance, one option is to have an input whereby the distance from the lesion to the sensor traverse start point can be quite separately measured and then input into the device. In this way the device will have information as to how far from the lesion scanning starts, i.e. 20 mm or perhaps 60 mm (0.8 in or 2.5 in). The device then knows, when the scan data is displayed, which section refers to the line before the lesion begins. This information is important, as it is common for the thermal profile of a melanoma to extend anything up to 10 cm (4 in) from the lesion, though usually the extent is much less, in the order of 2-6 cm (0.8-2.5 in).

Another method of sensor traversing involves combining the sensor with an “electronic ruler”. An example of such a ruler has two separable callipers, and their separation is measured by the ruler. In use, one calliper is held on the skin, and is stationary, whilst the other, holding the sensor, is moved over the skin and skin lesion, in a straight line. A cog-and-wheel arrangement facilitates the movement of the sensor housing calliper along the ruler, and the distance is monitored electronically. The movement of the sensor along the ruler can be made by incorporating a linear movement sensor such as a potentiometer, LVDT (linear velocity displacement transducer) or a cog to translate linear motion into circular motion, and a form of circular motion sensor, such as an optical encoder, potentiometer or synchro.

Again, the sensor's temperature readings are recorded sequentially with distance traversed along the ruler. The calliper height, and the height of the sensor, are adjustable.

The moveable calliper can be made to be flexible—one made out of rubber, for instance. The whole calliper may be flexible, or just the lower portion. In any event it can be made flexible enough to brush over the skin lesion, without causing the sensor to lose height.

This arrangement allows the sensor to be manually moved over the skin lesion in a straight line, at a fixed distance from the skin.

The speed of the sensor's traverse is not especially significant, but the data acquisition and computational arrangements will need to be able to cope with a range of speeds. And while the traverse may be a powered one, the apparatus being driven across the skin by a motor of some sort, it is quite practicable to employ hand-power to push the sensor along. For a hand-powered system, traverse speeds in the range 0.5 to 10 cm (0.2-4 in) per second should be expected.

The system of the invention incorporates means for sequentially recording the IR radiation incident on the sensor at a series of points along the line. Though with modern computing capability it may not matter much, provided the system acquires both sensor IR output data and sensor position data simultaneously, the points are conveniently at equal spacings along the traversed line (i.e. in general at equal time intervals with a constant speed of traverse). Depending on the type of traversing means employed, an encoder may be incorporated to provide data relating to the instantaneous position along the line where the infra-red radiation is sensed (it will be apparent that if a stepper motor is employed to drive the system then the use of an encoder is not essential).

The system's data recording means not only stores the data but also sends it on to the display means which shows a profile of the temperature along the sensed line over the lesion. This conveniently involves the use of a computer—a dedicated microprocessor controlling a suitable display device. The display means can take any appropriate form, but conveniently it is the video screen of either a separate desktop computer (a PC), a small hand-held computer, or the system's own built-in liquid crystal display (an LCD). The display is of the line profile—that is, it is a read-out based on the categories of thermal influence and relative temperatures, or simply a record of the temperature readings at different positions along the line.

While the gathered data may be first recorded and then—possibly some time later—be displayed, the display can of course be made in “real time”. In this respect the display will be updated continuously as the sensor moves and acquires data.

The system of the invention provides a line profile across the skin, including the area where this is a suspect skin lesion, and from this there can be deduced whether the lesion is likely to be malignant. The way the data is handled to achieve this end deserves further comment, now given.

As observed above, the system needs to know when to start recording, and very preferably needs to recognise different functional areas of the line it scans. Moreover, most preferably the system has the ability to recognise temperature gradients—this will enable it automatically to start recording, and will enable data analysis to be performed on the data recorded.

From the inventors' detailed analysis of skin lesions of many sorts the following general information is known.

The line temperature profile can be split into significant segments as follows:

Far from the lesion: normal skin, a relative low temperature that is relatively constant.

From the outer extent of thermal influence to the actual lesion: a gradient of raised and rising temperature.

Over the lesion: a sustained high temperature (but see below).

The temperature will decline on the other side of the lesion in a mirror image of these three stages.

Over the lesion the sustained high temperature might not be so uniform; there could be a high degree of variability in the area directly over and immediately around the lesion. Studies have shown the strong likelihood of a “coral atoll” effect, with a ridge of high temperature surrounding an inner core of relatively lower temperature. This “coral atoll” temperature profile is a characteristic that the system can be programmed to recognise.

The obtained line profile can in some instances be used to give the thermal profile a category dependent upon the area of thermal influence the skin lesion has, as well as its relative temperature reading. These categories can be as follows:

1) thermal influence area

<1 cm from lesion

1-2 cm from lesion

2-3 cm from lesion

3-4 cm from lesion

>4 cm from lesion

etc etc

2) differential temperature (lesion versus normal surrounding skin)

<0.5° C.

0.5-1° C.

1-2° C.

2-3° C.

3-4° C.

>4° C.

The system can be programmed to recognise these boundaries by setting gradient characteristics on the data.

One of the advantages of recognising temperature gradients is—as noted hereinbefore—that the system can decide when to start recording. It will recognise when the temperature gradient rises significantly, and thereupon start data recording. For instance, whilst being moved across normal skin, which will have a normal temperature variability, relatively speaking, no data will be recorded, but data acquisition will be triggered when the temperature rises significantly—for example, more than 0.2° C. over 1 cm (0.4 in). Of course, there can be an override to the automatic-start recording function, so that the Operator can manually start the system, and thus begin temperature recording and data display.

So, suitably programmed the system will have the following characteristics:

automatic start facility based upon the recognition of changing temperature profile

ability to recognise an area of raised temperature as the device moves from normal skin towards a suspect skin lesion

ability to recognise when the skin lesion has been reached, by virtue of the data held by itself regarding the initial distance from skin lesion inputted or measured by the device

ability to categorise the line over the area of thermal influence (from normal skin to lesion)

ability to recognise a “coral atoll” line profile over the suspect skin lesion

There will be circumstances where the skin lesion is analysed from more than one angle, and thus where the line taken over it is varied in position. The usual process would be to take line profiles that are at an angle of 90° to each other. The data from two such perpendicular line profiles can be amalgamated and displayed. One embodiment of the display would be to render a three-dimensional representation of the lesion thermally. A number of line profiles can be taken, at unique angles, over the skin lesion, say (for instance) at 30° intervals from 0° to 330°. A much better three dimensional profile of the lesion can then be given.

Several embodiments of the invention are now described, though by way of illustration only, with reference to the accompanying diagrammatic Drawings in which:

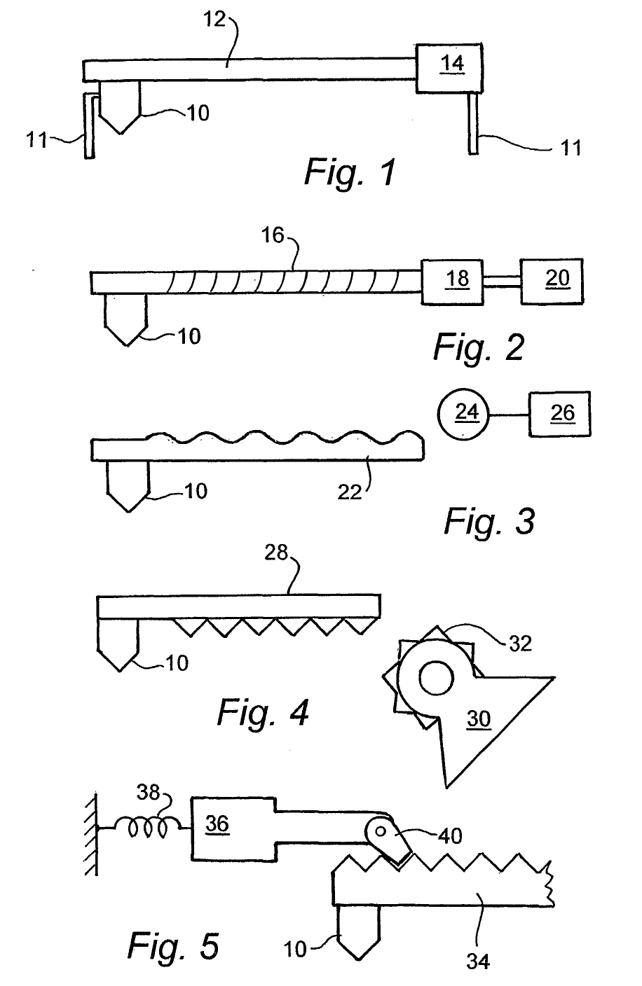

FIG. 1 shows a linear-traversing, skin-scanning infra-red sensor system of the invention; and

FIGS. 2-8 show other forms of linear-traversing, skin-scanning infra-red sensor system of the invention.

FIG. 1 shows a linear-traversing, skin-scanning infra-red sensor system of the invention. In the arrangement of FIG. 1, the system has a housing (10) on which is mounted an IR sensor (not shown separately). The housing/sensor is carried by a lead screw (12) driven by a stepper motor (14). As the lead screw 12 turns so the housing/sensor 10 travels out (and then back) along the screw, passing in a straight line over the area of skin (not show) to be investigated. Heat radiation from the skin over which the sensor 10 passes is received by the sensor and then passed back (by means not shown) to a recording and display device (not shown).

The sensor system shown in the Figure has two touch rods (as 11) for assisting in measuring, and maintaining, the sensor the required distance from the skin. One touch rod is mounted on the sensor housing 10, while the other is mounted—at the other end of the apparatus—on the stepper motor 14. Both are adjustably mounted (not shown), so enabling the distance of the sensor to be both set and maintained while it travels along the path determined by the lead screw 12.

FIG. 2 shows a first alternative version of the FIG. 1 system. In this alternative, a continuous drive to a lead screw (16) carrying the sensor 10 is applied by a motor (18) having an associated encoder (20). In use the signals received from the sensor 10 are “combined” with signals output from the encoder 20 to provide accurate time/distance references for the display system (not shown).

In the arrangement of FIG. 3, the sensor 10 is carried by a linear rack (22) engaged by a pinion (24) driven by a motor (26).

FIG. 4 shows an arrangement where the sensor 10 is mounted at the end of a linear ratchet (28) powered by a trigger arrangement (30) including a toothed wheel (32), such that repeatedly pressing the trigger (not shown) turns the wheel 32 and so drives the ratchet 28 out, thus moving the sensor 10 over the skin.

In FIG. 5 there is shown an arrangement employing a sensor-carrying toothed linear bar (34) powered by an electromagnet (36) and an associated return spring (38). When the magnet 36 is powered it pushes on a hinged pawl (40) engaging the linear bar 34, pushing the bar away and allowing a linear movement of one tooth when the powering current flow stops. Thus, the supply of a pulsed current to the electromagnet 36 produces a stepped linear movement of the bar 34 carrying the sensor 10.

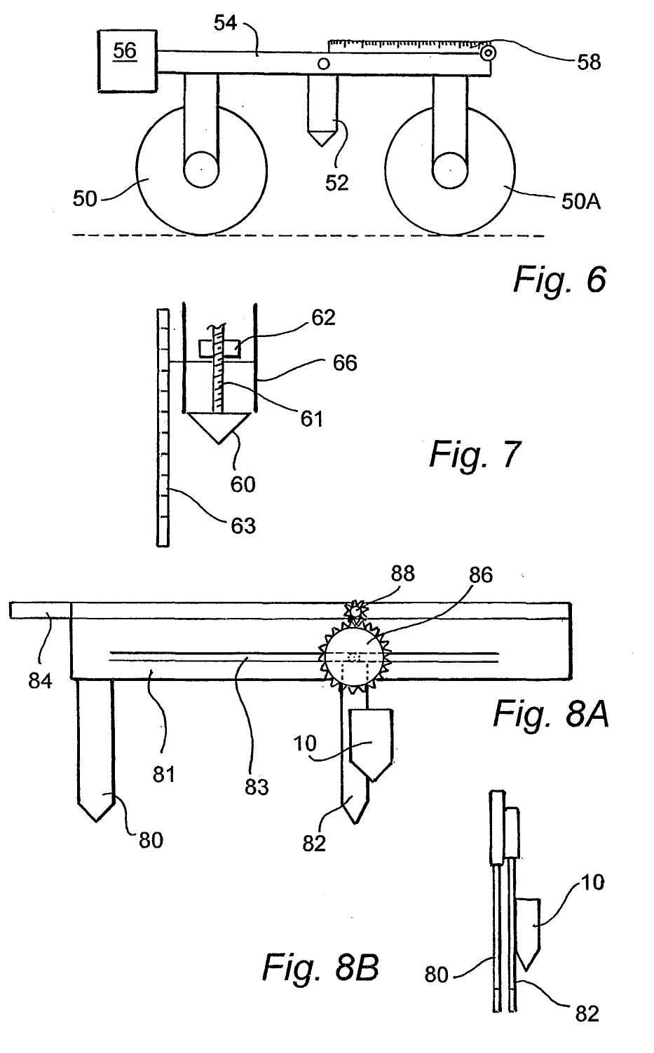

Another method of arranging the traversing mechanism by which the infra-red sensor may be moved in a line over the suspect lesion is shown in FIG. 6. This depicts a cradle (54) having (in this case) two wheels (50,50a) the latter of which is coupled (by means not shown) to a rotary motion measurement means (56), the cradle carrying the IR sensor (52) beneath it. Thus, the sensor 52 is housed in the cradle 54 that runs on the wheels 50.

The wheels 50 ensure that a fixed distance from the skin is maintained as the cradle 54 is moved in a line across the skin over the suspect lesion. There is no requirement for the traversing mechanism to be powered, except by hand.

The rotary motion sensor 56 is coupled to the wheel mechanism so that the readings of the infra-red sensor 52 are taken at fixed and pre-set intervals of distance. In this way readings of the infra-red sensor 52 can be taken sequentially along the line over the skin which includes the suspect skin lesion, maintaining the sensor over the skin.

A ruler (58) is carried on the cradle 54 hingedly secured to one end (the right, as viewed) so that it can be unfolded and used to measure the distance from the scan start point to the suspect skin lesion.

FIG. 7 shows a detail of a sensor mounting useable in any of the systems of the invention, but typically in one such as in FIG. 6.

The sensor (60) is carried at the end of a housing (66) in which it is mounted by means of a threaded rod (61) and a corresponding adjustment nut (62). A touch rod (63) is secured to the side of the housing 66 to enable there both to be taken a measurement of the height of the sensor 60 and to be maintained that height as the sensor travels across the surface of the skin (not shown).

FIGS. 8A and 8B show a device of the invention employing an “electronic ruler” (FIG. 8A shows a front view while FIG. 8B shows a side view).

This embodiment of the device has an elongate body (81) at one end of which is mounted a fixed calliper (80). From that end, at which there is also mounted a linear movement sensor (84), there extends a slot (83), and mounted for movement along that slot is a movable calliper (82) which can be driven back and forth along the slot by a cog and wheel arrangement (86,88). Carried by the movable calliper 82 is the sensor housing 10 (the sensor is not separately shown).

In all the cases shown in the Drawings, sequential movements taken by the IR sensor as it traverses a straight line over a suspect lesion, at a fixed distance above the skin, are fed to a microprocessor, which may also control operation of the traversing means, whereby to produce a linear temperature profile on a suitable display device such as an LCD.

1. A system for detecting skin and sub-surface malignancies, the system comprising:
an infra-red sensor positionable adjacent the skin, at a given distance above the skin, to detect infra-red radiation emanating from the skin and a suspect lesion therein;
means for traversing the sensor in a measurable manner to scan along a line across the skin and the lesion therein;
means that gauges the distance from where the scan starts to the lesion;
means for sequentially recording the output of the sensor, and thus infra-red radiation incident on the sensor, at a series of points along the line; and
means for displaying a profile of the temperature along the sensed line over the lesion, from which profile there can be drawn a conclusion about the presence or absence of a malignancy.

2. A system as claimed in claim 1, wherein the infra-red sensor is a thermopile.

3. A system as claimed in either of the preceding claims, wherein the infra-red sensor as associated therewith IR-transparent optical means to focus the infra-red radiation on to it.

4. A system as claimed in any of the preceding claims, wherein the IR-sensor is held by a housing itself positioned a small fixed (and known) but adjustable distance above the skin surface, there being means for guiding the sensor/housing to maintain the required distance above the skin as it traverses the lesion.

5. A system as claimed in claim 4, wherein the guide means is a skin touch rod mounted relative to which the sensor is adjustably mounted.

6. A system as claimed in any of the preceding claims, wherein the sensor height adjustment arrangement is able to cope with varying heights of lesion.

7. A system as claimed in any of the preceding claims, wherein the height adjustment arrangement is such that the sensor housing is adjustable prior to use, and its height is set for the duration of scanning.

8. A system as claimed in any of claims 1 to 6, wherein the sensor housing automatically adjusts itself so that the distance between sensor and skin surface remains constant, utilising an automatic focus system to ensure that the sensor is optimally focused on the skin surface.

9. A system as claimed in any of the preceding claims, wherein the means for traversing the sensor in a measured manner along a line across the skin is one or more wheel to which the sensor and its associated equipment is suitably mounted.

10. A system as claimed in any of the preceding claims, wherein the system's means for sequentially recording the IR radiation incident on the sensor at a series of points along the line include an encoder to provide data relating to the instantaneous position along the line where the infra-red radiation is sensed.

11. A system as claimed in any of the preceding claims, wherein the display means to which is sent the data acquired by the recording means is a dedicated microprocessor controlling a suitable display device.

12. A system as claimed in any of the preceding claims, wherein the profile display means is associated with computing and calculating means able to provide an indications as to whether the lesion is likely to be malignant.

13. A system as claimed in claim 12, wherein the computing and calculating means is able to recognise temperature gradients, and from this is enabled automatically to start recording, and thereafter to carry out the required analysis of the data recorded.

14. A system as claimed in any of the preceding claims and substantially as described hereinbefore.

15. A method for detecting skin and sub-surface malignancies, in which method, and using apparatus as defined in any of the preceding claims:
an infra-red sensor is positioned adjacent the skin, at a given distance above the skin, to detect infra-red radiation emanating from the skin and a suspect lesion therein;
the sensor is caused to traverse in a measurable manner along a line across the skin and the lesion therein, there having been gauged the distance from where the scan starts to the lesion;
there is sequentially recorded the output of the sensor, and thus infra-red radiation incident on the sensor, at a series of points along the line; and
there is displayed a profile of the temperature along the sensed line over the lesion, from which profile there can be drawn a conclusion about the presence or absence of a malignancy.

16. A method as claimed in claim 15, in which the line which the sensor traverses is a straight line extending from a point just at one side of the lesion to a point just at the other side.

17. A method as claimed in either of claims 15 and 16, in which the distance from the lesion to the sensor traverse start point is quite separately measured and then input into the device.

18. A method as claimed in any of claims 15 to 17, in which the IR radiation incident on the sensor is recorded at a series of equispaced points along the line.

19. A method as claimed in any of claims 15 to 18, in which the system is programmed to recognise and categorise a line temperature profile split into significant segments—far from the lesion, from the outer extent of thermal influence to the actual lesion, and over the lesion.

20. A method as claimed in claim 19, in which the system is further programmed to recognise the “coral atoll” effect, with a ridge of high temperature surrounding an inner core of relatively lower temperature.

21. A method as claimed in any of claims 15 to 20, in which the skin lesion is analysed from more than one angle, and thus where the line taken over it is varied in position.

22. A method as claimed in claim 21, in which lines are at an angle of 90° to each other.

23. A method as claimed in any of claims 15 to 22 and substantially as described hereinbefore.
